(19) United States
(12) Patent Application Publication
(10) Pub. No.: US 2015/0099057 A1
(43) Pub. Date: Apr. 9, 2015

(54) COMPOSITION OF MATTER FOR REPELLING MOISTURE FROM A DENTAL MIRROR AND PROCESS FOR APPLYING THE SAME

(71) Applicants: RICHARD JOHNSON BARTLETT, HOUSTON, TX (US); WILLIAM TRAVIS BARTLETT, HOUSTON, TX (US)

(72) Inventors: RICHARD JOHNSON BARTLETT, HOUSTON, TX (US); WILLIAM TRAVIS BARTLETT, HOUSTON, TX (US)

(21) Appl. No.: 14/509,329

(22) Filed: Oct. 8, 2014

Related U.S. Application Data
(60) Provisional application No. 61/877,664, filed on Oct. 8, 2013.

(51) Int. Cl. C09K 3/18; A61B 1/253

(52) U.S. Cl. CPC C09K 3/18; A61B 1/253
USPC 427/2.29; 106/2

(57) ABSTRACT

A composition of matter may be effective for repelling spray and condensation of aerosol moisture, such as breath, from a desired surface, such as a mirror. The composition of matter may include a hydrophobic admixture of one or more of at least one terpene, at least one alcohol, at least one ester and at least one wax. The admixture containing the at least one terpene, the at least one alcohol, and the at least one ester combined with the at least one wax may be used to produce a hard coating on the desired surface. Polishing the hard coating may further enhance the water repulsion efficacy of the coating.

### RELATED APPLICATION

This application claims priority to provisional patent application U.S. Ser. No. 61/877,664 filed on Oct. 8, 2013, the entire contents of which is herein incorporated by reference.

### BACKGROUND

The embodiments herein relate generally to processes for treating a glass or other desired surfaces to repel water. In particular, the embodiments herein relate to processes for treating a mirror with substances to repel water which are medically safe for human oral and respiratory exposure.

A common, critical instrument used by all dentists and dental assistants is the diagnostic mirror, sometimes called the examination mirror. This mirror enables dental care providers to view areas of dental structures within a patient's mouth that are not visible via direct observation. However, using a dental mirror is problematic because the mirror image is often obscured by accumulated droplets from instrument spray or by clouding from the condensed moisture in the breath of a patient. Both of these sources produce either droplets or a condensate that adhere to the mirror, which in turn distorts the images on the reflective surface of the mirror. This image distortion on the mirror's reflective surface necessitates that the mirror be cleaned to clear the reflective surface. The dentist or dental assistant must interrupt the procedure, remove the mirror from the mouth and wipe off the adherent moisture with a sterile tissue. This can be quite disruptive to dental procedures, wastes time, and requires certain precautions be taken to avoid introducing infectious agents into the patient's mouth. Dental professionals also use intra-oral (palatal) mirrors for their photography records. To compensate for the fog resulting from a patient's breath, dentists use a specialized device in which the mirrors are given a warm bath to momentarily prevent the fog buildup. This is impractical for regular dental exam mirrors and is no better than repeated wiping of the mirrors. If adherence of moisture to the reflective surface of the either examination or intra-oral mirrors could be prevented by using a coating for the mirrors approved for human use, then the whole process of dental procedures will become more efficient, safer, and thus less costly.

Some other endeavors in this field include: U.S. Pat. No. 6,296,694 issued to Machson; U.S. Pat. No. 3,902,245 issued to Wolf; U.S. Patent Application 2011/0033818 filed by Miller; U.S. Pat. No. 5,716,921 issued to Neumiller; U.S. Patent Application 2006/0135649 filed by Jedlicka; U.S. Pat. No. 4,343,725 issued to Kiewert; U.S. Pat. No. 2,353,978 issued to Weber; U.S. Pat. No. 4,400,157 issued to Moore; U.S. Pat. No. 6,257,888 issued to Barham; U.S. Pat. No. 7,325,675 issued to Halkyard; U.S. Pat. No. 7,455,519 issued to Barstow; U.S. Pat. No. 3,393,416 issued to Kilpatrick; Great Brittan Patent 611,624 issued to Freeman; U.S. Pat. No. 3,755,903 issued to Spinello; Kerr Dental Mirror spray is available at http://www.kerrdental.eu/catalog-files/1/1104/files/MSDS_Mirror_Spray_en-US.pdf.

There are two seemingly independent lines of research in dental mirror defogging and spray removal. In the first line, a layer of plastic is impregnated with some compound, usually a wetting agent, and applied to the mirror. Spinello, Kerr, Barham, Miller, Kiewer, Jedlicka, Newmiller, Machson are in this field. The second field focuses on ways of applying benzalkonium chloride, commonly sold under the name Butler Clear Dip ®, to a mirror with some system. Freedman, Barstow, Halkyard, Kilpatrick and Moore are in this group. There are two outliers: Weber teaches a cotton applicator using wax and a fatty acid and Wolf seemingly stumbled upon baking soda and corn starch as a purportedly effective composition of matter.

Spinello teaches an anti-misting attachment for dental mirrors. The attachment can be formed of a plastic such as an acrylic which is polymerized in the presence of a surfactant, thereby entraining minute quantities of the surfactant throughout the plastic. Spinello found polyoxyethelene cetyl ether to be an adequate surfactant to operate as a plastic. Kerr is similar, teaching an alcohol combined with a plastic. Its composition of matter including: Isopropyl alcohol, Trideceth 8, Propane, n-Butane, Iso-Butane, while 1, 3 Butadiene to forms the plastic coating. Barham advocates for a hydrophilic polyurethane foam seemingly to operate as a sponge. Miller likewise appreciates the sponge, though the sponge would be impregnated with a disinfectant, detergent, enzymatic cleaner or hydrophilic surfactant. Kiewert likewise uses a plastic comprising a nonionic polyoxyethylene glycol polymer with water-soluble, synthetic surface-active compound selected from the group consisting of synthetic anionic surface-active compounds, nonionic surface-active compounds, cationic surface-active compounds, and compatible mixtures thereof and an alkaline-reacting compounds selected from the group consisting of alkali metal and ammonium hydroxides, alkali metal and ammonium carbonates, alkali metal and ammonium bicarbonates. Jedlicka also uses a plastic base such as polyurethanes, polyesters, epoxy resins, and polyacrylates combined with hydroxyl group-containing fatty acid esters of polyhydric alcohols. Neumiller utilizes a four part compound with an alkyl or fatty acid, 2-hydroxyethyl or (poly)ethoxyethanol, an organic moiety, and halogen, methyl sulfate or ethyl sulfate. Machson applies a surface agent containing sodium lauryl ether sulfate, and either a block ethylene oxide/polyethylene oxide copolymer, an ethoxylated amine, an ethoxylated acetylenic alcohol, sodium sulfosuccinate, ethoxylated sorbitan ester, random EO/PO polymers on butyl alcohol.

All of the above embodiments rely on hydrophilic components contained within the plastic matrix, which itself, will have components that may or may not be completely safe for human oral or respiratory exposure.

Most of the traditional work in this field focused on modifying dental instruments themselves to repel fog. For instance, Freedman teaches a supplementary vacuum attachment to remove excess spray, but has no theory as to fog from breath. Barstow teaches a wiper on the mirror itself. Halkyard takes this a step further and applies a backing strip to the mirror with a wiper to apply benzalkonium chloride. Kilpatrick teaches both a machine for holding an instrument in a solution of Butler Clear Dip ® which, in according to Kilpatrick, relied on benzalkonium chloride as its active ingredient, the present composition of matter, however, is a trade secret. Moore teaches a system for dispensing droplets of benzalkonium chloride (which it calls dimethyl benzyl ammonium chloride). Benzalkonium chloride has known toxic risks for oral and respiratory exposure.

Weber teaches a few different kinds of compositions of matters to prevent fogging of glass, including impregnating paraffin wax, beeswax, carnauba wax, Montana wax with hydrophilic fatty acids and some of which may be attached to or derived from glycol and glycerin, all highly soluble in water. Moreover, Weber teaches the use of hydrophilic alkylated naphthaline sulfonic acids, alkylated phenol sulfonic acids, both of which have known oral and respiratory toxicity, rather than using hydrophobic terpenes. Weber relies on calcium and magnesium salts all of which are readily soluble in water. Wolf operates on another level and teaches an aqueous mixture of baking soda and corn starch.

### SUMMARY

A composition of matter is effective for repelling spray and breath from a mirror or other desired surfaces. The composition of matter comprises a hydrophobic combination of one or more of at least one terpene, at least one alcohol, at least one ester, and at least one wax. The admixture is heated such that the at least one or more terpene, the at least one or more alcohol, and the at least one ester combine with the at least one wax or saponified wax to produce a hard coating on the mirror.

While the individual components of the admixture described below have efficacy for resisting water, combinations of the terpene, alcohols and/or esters have additive efficacy. In some embodiments, the admixture base begins with at least one cyclic terpene, which is limonene. In some embodiments, the at least one alcohol is at least one member selected from the group consisting of: ethanol, octanol, undecanol, dodecanol, cetyl alcohol, octyldodecanol, and nonadecanol. In some embodiments, the at least one alcohol is at least one member selected from the group consisting of: 1-Tetracosanol, 1,2-dodecanadiol, 1-hexacontanol, 1-nonadecanol, 1-lignaceryl, and 1 triacontanol, wherein the at least one alcohol is dissolved in the admixture. In some embodiments, the ester is ethyl oleate. In some embodiments, the admixture further comprises a base alcohol-ester mixture having three parts by volume of the saponified wax for each part of the alcohol-ester admixture composed of at least the base of limonene, with added octanol, undecanol, dodecanol, and ethyl oleate. In some embodiments, the admixture further comprises a base alcohol-ester mixture having four parts of the saponified wax for each part of the limonene-alcohol-ester admixture above. In some embodiments, the composition further comprises at least one member selected from the group consisting of the class of: 1,2-dodecanadiol, 1-hexacontanol, 1-nonadecanol, 1-lignaceryl, and 1 triacontanol dissolved in the terpene-alcohol-ester admixture. In some embodiments, this terpene/alcohol/ester admixture is applied directly to the mirror and allowed to air-dry. In some embodiments, the composition further comprises a non-ionic surfactant, such as lecithin. In some embodiments, the wax is at least one member selected from the group consisting of paraffin wax and carnauba wax. In some embodiments, the paraffin wax and the carnauba wax are both present in a range of ratios from one to five parts by volume of paraffin wax for each part of carnauba wax.

A process for applying a hydrophobic coating onto a mirror contains the following steps, which are not necessarily in order. First, the at least one wax may be extracted with filtered water at about 100° C. to remove any aqueous soluble components, thus creating an aqueous phase and a wax phase in suspension at about 100° C. The wax phase may be used directly to create the admixture for application to the mirror or may be further saponified by adding aqueous sodium hydroxide, creating an aqueous phase and a wax phase in suspension at about 100° C. Then, the aqueous phase may be removed and the wax phase may be re-extracted through adding filtered water at about 100° C. After that, the wax phase may be mixed with an admixture of at least one terpene, at least one alcohol, and at least one ester. Following that, the wax-admixture may be applied onto a mirror. Next, the wax-admixture may be directly polished onto the surface of the mirror, or the wax-admixture phase may be heated on the mirror and then the wax-admixture may be polished onto the mirror surface. The wax-admixture may then be allowed to cool and solidify on the mirror surface.

### BRIEF DESCRIPTION OF THE FIGURES

The detailed description of some embodiments of the invention is made below with reference to the accompanying figures, wherein like numerals represent corresponding parts of the figures.

FIG. 1 shows a flowchart of an embodiment of the invention.

FIG. 2 shows a flowchart of an embodiment of the invention.

### DETAILED DESCRIPTION OF CERTAIN EMBODIMENTS

Terpenes are unsaturated compounds formed by joining together isoprene units. When isoprene units are linked in rings, a “cyclic terpene” is formed. For example, limonene, camphor, menthol, carvone, terpineol, alpha-lonone and Thujone are cyclic terpenes.

An alcohol is an organic compound in which a hydroxyl functional group (—-OH) is bound to a carbon atom. For example, ethanol, octanol, undecanol, dodecanol, cetyl alcohol, octyldodecanol, and nonadecanol are alcohols.

Turning first to non-fatty alcohols, an octanol is an 8-carbon alcohol water insoluble liquid. There are 89 possible isomers of octanol. Ethanol is a volatile, flammable, colorless liquid with the structural formula CH₃CH₂OH, often abbreviated as C₂H₅OH or C₂H₆O. Undecanol is a colorless, water insoluble liquid having a melting point of 19° C. and a boiling point of 243° C.

Fatty alcohols (or long-chain alcohols or organic alcohols) are usually high-molecular-weight, straight-chain, primary alcohols, but can also range from as few as 4-6 carbons to as many as 30 or more, and may be derived from natural fats and oils. Dodecanol is a fatty alcohol that is a colorless solid with a floral smell. Cetyl alcohol, also known as 1-hexadecanol and palmityl alcohol, is a fatty alcohol with the formula CH₃(CH₂)₁₅OH. The diol 1,2-dodecanadiol is a fatty alcohol with two —OH functional groups attached to adjacent carbons and takes the form of a waxy white solid or flakes at room temperature. At room temperature, cetyl alcohol takes the form of a waxy white solid or flakes. Octyldodecanol is a fatty alcohol appearing as a clear colorless liquid at room temperature. Nonadecanol is a fatty alcohol appearing as a white crystalline powder at room temperature.

1-Tetracosanol is a fatty alcohol, usually derived from the fatty acid Lignoceric acid. Triacontanol is a fatty alcohol of the general formula C₃₀H₆₂O, also known as melissyl alcohol or myricyl alcohol. It is found in plant cuticle waxes and in beeswax.

An essential oil is a concentrated hydrophobic liquid containing volatile aromatic compounds extracted from plants. The following essential oils were used in experiments later discussed: orange essential oil, Tangerine essential oil, lime essential oil, lemon essential oil, pink grapefruit essential oil, mandarin essential oil, marjoram essential oil, ravintsara (camphor) essential oil, hemp essential oil and Geranium (Rose) essential oil. Of course, other essentials oils may be used. In many cases, the major hydrophobic component of most of these essential oils is d-limonene, being as high as 95% in some cases. Where d-limonene is not present, other terpenes with similar chemical structures are present.

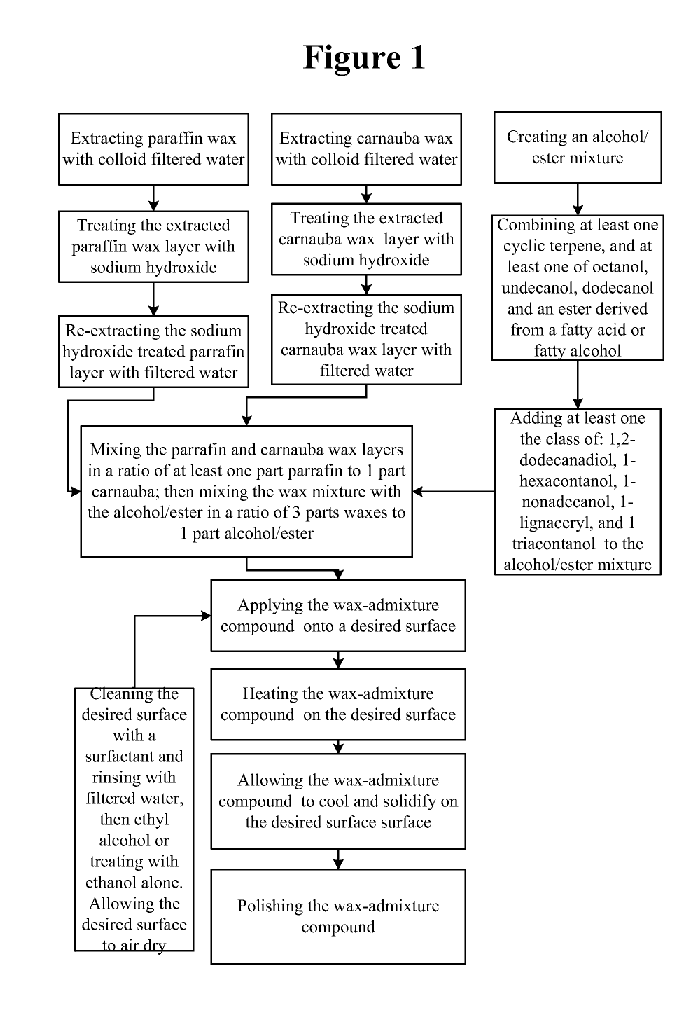

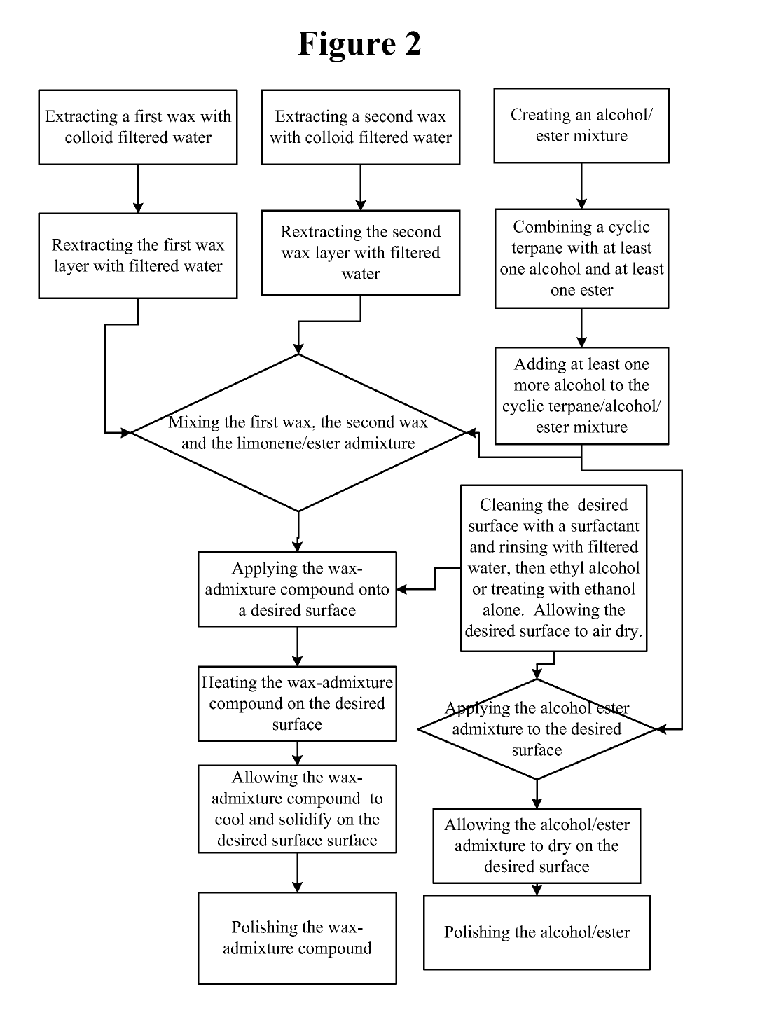

By way of example, an as shown in FIG. 1 and FIG. 2, a process for manufacturing a composition for repelling moisture from a dental mirror and for applying the composition to a dental mirror may include the following steps. First, the at least one wax may be extracted with filtered water at about 100° C. to remove any aqueous soluble components, thus creating an aqueous phase and a wax phase in suspension at about 100° C. The wax phase may be used directly to create the admixture for application to the mirror, or it may be further saponified by adding aqueous sodium hydroxide, creating an aqueous phase and a wax phase in suspension at 100° C. Then, the aqueous phase may be removed and the wax phase may be re-extracted through adding filtered water at about 100° C. After that, the wax phase may be mixed with an admixture of at least one terpene, at least one alcohol, and at least one ester. Following that, the wax-admixture may be applied onto a desired surface. The wax-admixture may be directly applied and polished on the desired surface, or the wax-admixture phase may be heated after application on the desired surface, allowed to solidify, and then the solidified wax-admixture may be polished onto the mirror surface.

Carnauba wax produces a hardened coating with a penetration of 2 dmm at 250 degrees Centigrade and 3 dmm at 43.30 degrees Centigrade. The carnauba wax layer retained these properties throughout the various extraction protocols. At room temperature in all admixtures, these wax components produced a coating resistant to the friction of polishing of the coating leaving a surface impervious to water. In some cases, this solidified admixture could be rubbed onto the mirror surface in semi-solid form, while other cases required heat to spread the admixtures onto the treated mirror.

Experiment 1: Wax Selection

Paraffin wax and carnauba wax were tested separately and together. Beeswax could also be used, but the current strategy relied on using the identified fatty alcohols from beeswax to be certain that bee venom or other allergens would not be present in trace amounts in preparations of beeswax. The carnauba wax added a slight yellow tint, leading to a process of saponification, or a bleaching protocol, which removed both glycerol and other water soluble components, as well as converting the remaining compositions of matter into primary alcohols and esters.

Saponification of mixtures of paraffin and carnauba began by heating the wax mixture to 105° C. and extracting the wax by addition of filtered or medical grade water heated to 100° C. The sharp interface that formed allowed removal of the aqueous phase from underneath the wax phase. As used in this application the “sharp interface” is the interface that is produced by allowing the wax layer to come to room temperature where the wax layer becomes solid. In a test tube this usually leaves a central column that is free of the wax and by pushing the wax layer towards the bottom of the tube the water is removed by simply pouring it out. The sharp interface is the solid wax versus the liquid aqueous layer.

In some varieties of this experiment, the water-extracted wax phase was used directly for creating admixtures. In others, further saponification of the wax phase was by adding an equal volume of 100° C. 0.05 molar NaOH and mixed thoroughly. This mix was allowed to remain overnight at 95° C. until complete phase separation had occurred. Again, the sharp interface that formed allowed removal of the aqueous phase from underneath the organic phase. Water extraction was repeated as above at least twice. The aqueous phase was tested for pH, which generally approached 7.5 within two extractions at 95° C. After the final aqueous extraction, the organic phase was removed and transferred to a fresh tube for storage. Other saponification methods may be used, but these processes have in common the removal of aqueous components to produce a highly hydrophobic wax layer useful for the invention described herein.

Experiment 2: Admixture Selection

Mirrors were cleaned with absolute ethanol, wiped with a lens quality microfiber paper and the ethyl alcohol was allowed to evaporate briefly at room temperature, which is about 20 to 26 degrees Centigrade. Individual coating components were initially tested as pure compounds. To test water-repelling capacity, mirrors were coated by aliquoting enough hydrophobic sample to coat the mirror (about 15 to about 30 μl) of either pure compound or the different admixtures onto the mirror surface. The wax-admixtures were polished directly or spread by heat aimed at the mirror and allowed to air-dry overnight. The length of air-drying time was not found to be critical once the admixture hardened.

Pure limonene was found to be an effective base-sheeting component, which substituted for many of the essential oils for all admixtures. As ethoxylates are major components of cosmetics, these were also tested, but the addition of ethoxy moieties to fatty alcohols added some hydrophilic nature to the admixtures. Thus, while included in separate testing and as a component with each individual fatty alcohols and wax admixtures, ethoxylates allowed for water retention on mirrors. It was found that addition of low molecular weight organic alcohols (less than 8 carbons), which had some solubility in water, also allowed for water retention on mirrors. Consequently, naturally occurring, higher molecular weight fatty alcohols (either United States Pharmacopeia (USP) pure, generally recognized as safe, (GRAS) or food grade) found as either components or natural metabolic by-products of food stuff were selected for testing based on being non-miscible with water and existing in natural products accepted for human exposure. Fatty alcohols, existing as solids at room temperature, were dissolved in limonene or an alcohol admixture at a concentration of at least about 10 mg/ml. Many of these higher molecular weight alcohols or fatty alcohols were found to augment sheeting as well as add to resistance of condensation from breath over and above the effects of limonene alone.

Experiment 3: Water Repulsion

To test water repulsion, treated, air-dried mirrors were tested for resistance to breath condensation by multiple exhaled breaths, wherein the mirrors were held less than about 2 cm from the mouth. Repeated tests were averaged to determine resistance to condensation. Testing of repulsion of spray to saturation was completed using a spray bottle filled with colloid filtered water. Success was deemed the lack of water retention on the surface of the mirror (sheeting). Once admixtures were deemed successful, the final tests were repeated and digitally recorded.

A summary of the testing results found that an admixture of d-limonene, fatty alcohols and esters, and saponified waxes gave optimal water repulsion leaving mirrors with non-distorted image viewing. Testing found that wax-containing admixtures worked best when applied with heat, and once the wax admixture was liquid, microfiber wipes were used to polish the admixture onto the mirror, then air-dried. Treated mirrors were polished with the lens quality microfiber paper or cloth after initial exposure to spray. However, any good quality paper or cloth or even a buffing wheel that does not leave a lint residue would suffice for this polishing step. As used in this application a “good quality paper” is one that does not leave a lint residue. The exposure to water spray followed by polishing may produce a very thin, extremely hard coating, which acts as a hydrophobic shield to the mirror due to the very hard hydrophobic components in the carnauba wax. On a hard surface, such as a treated mirror, the limonene and alcohols imbedded in the hardened wax components may be retained on the mirror throughout testing. Wax mixtures with added limonene produced excellent resistance to breath, but generally poor sheeting, causing spray to bead on the surface when treated mirrors were not first polished. Admixtures of limonene and fatty alcohols produced excellent sheeting of spray and resistance to breath. The combination of the two components may be ideal to optimize resistance to both breath and spray and retain the admixture components on the mirror during testing and re-testing.

Persons of ordinary skill in the art may appreciate that numerous design configurations may be possible to enjoy the functional benefits of the inventive systems. Thus, given the wide variety of configurations and arrangements of embodiments of the present invention the scope of the invention is reflected by the breadth of the claims below rather than narrowed by the embodiments described above.

Persons of ordinary skill in the art may appreciate that the functional benefits of the inventive systems may also be applied to desired surfaces other than mirrors. A desired surface is a surface that one intends to apply a compound upon. It can be a mirror.

What is claimed is:

1. A composition of matter, effective for repelling spray and condensation of aerosol moisture, from depositing on a desired surface; the composition of matter comprising:
an admixture comprising at least one terpene, at least one alcohol, at least one ester, which is directly applied to the desired surface;
at least one wax is heated and mixed with the admixture forming a wax-admixture compound,
wherein the wax-admixture compound is configured to be applied onto the desired surface to produce a hydrophobic coating.

2. The composition of matter of claim 1, wherein the at least one terpene is at least one cyclic terpene.

3. The composition of matter of claim 2, wherein the at least one cyclic terpene is limonene.

4. The composition of matter of claim 1, wherein the at least one alcohol is at least one member selected from the group consisting of ethanol, octanol, undecanol, dodecanol, cetyl alcohol, octyldodecanol, and nonadecanol.

5. The composition of matter of claim 1, wherein the at least one alcohol is at least one member selected from the group consisting of 1-Tetracosanol, 1,2-dodecanadiol, 1-hexacontanol, 1-nonadecanol, 1-lignaceryl, and 1 triacontanol, wherein the at least one alcohol is dissolved in the admixture.

6. The composition of matter of claim 1, wherein the at least one ester is ethyl oleate.

7. The composition of matter of claim 1 wherein the admixture further comprises a base limonene-alcohol-ester admixture having at least one part by volume of the at least one wax for each part of the admixture which is composed of a base of limonene, mixed with at least one member selected from the group consisting of octanol, undecanol, dodecanol, and ethyl oleate.

8. The composition of matter of claim 1, further comprising at least one member selected from the group consisting of a class of 1,2-dodecanadiol, 1-hexacontanol, 1-nonadecanol, 1-lignaceryl, and 1 triacontanol dissolved in the admixture.

9. The composition of matter of claim 1, further comprising a non-ionic surfactant.

10. The composition of matter of claim 1, wherein the at least one wax is derived from at least one of a paraffin wax and a carnauba wax.

11. The composition of matter of claim 10, wherein the paraffin wax and the carnauba wax are both present in a ratio of at least one part by volume of the paraffin wax for each part of the carnauba wax.

12. The composition of matter of claim 10, wherein saponification of the at least one of the paraffin wax and the carnauba wax produce hydrophobic fatty alcohols and esters and removes aqueous soluble components from the at least one wax.

13. A process for applying a hydrophobic coating onto a desired surface, the process comprising:
applying the admixture directly to the desired surface.

14. A process for applying a hydrophobic coating onto a desired surface, the process comprising:
forming a non-aqueous wax phase by removing aqueous soluble components from at least one wax by first extracting with water, and re-extracting the non-aqueous wax phase with additional water forming an extracted phase;
mixing the extracted wax phase with an admixture of at least one terpene, at least one alcohol, and at least one ester; forming a wax-admixture compound; and applying the wax-admixture compound onto the desired surface.

15. The process of claim 14, further comprising:
directly rubbing the wax-admixture compound onto the desired surface.

16. The process of claim 15, further comprising:
heating the wax-admixture applied to the desired surface to produce a more uniform coating.

17. The process of claim 16, further comprising:
polishing the wax-admixture compound.

18. The process of claim 14, further comprising;
heating the wax-admixture compound and applying to the desired surface;
allowing the wax-admixture compound to cool and solidify on the desired surface creating a solidified wax-admixture compound; and polishing the solidified wax-admixture compound.

19. The process of claim 18, further comprising:
heating the wax-admixture applied to the desired surface to produce a more uniform coating.

20. The process of claim 19, further comprising:
polishing the wax-admixture compound.

21. A process for applying a hydrophobic coating onto a desired surface, the process comprising:
forming an non-aqueous wax phase by removing aqueous soluble components from at least one wax by extracting with a water,
saponifying a non-aqueous wax phase in aqueous sodium hydroxide and re-extracting with water producing a saponified wax phase;
mixing the saponified wax phase with an admixture of at least one terpene, at least one alcohol, and at least one ester; forming a saponified wax-admixture compound; and
applying the saponified wax-admixture compound onto the desired surface.

22. The process of claim 21, further comprising:
directly rubbing the saponified wax-admixture compound onto the desired surface.

23. The process of claim 21, further comprising:
heating the wax-admixture applied to the desired surface to produce a more uniform coating.

24. The process of claim 23, further comprising:
polishing the wax-admixture compound.

25. The process of claim 21, further comprising;
heating the saponified wax-admixture compound and applying to the desired surface;
allowing the wax-admixture compound to cool and solidify on the desired surface; creating a solidified, saponified wax-admixture compound and
polishing the solidified, saponified wax coating.

26. The process of claim 25, further comprising:
heating the wax-admixture applied to the desired surface to produce a more uniform coating.

27. The process of claim 26, further comprising:
polishing the wax-admixture compound.

28. A process for applying a hydrophobic coating onto a desired surface, the process comprising;
applying an effective amount of a hydrophobic compound to the desired surface creating a hydrophobic coating on the desired surface.

29. The process of claim 28, where the hydrophobic compound includes one member selected from the group consisting of: a terpene, a fatty alcohol, an ester, and a wax.
